(19) United States
(12) Plant Patent Application Publication
(10) Pub. No.: US 2023/0037288 P1
(43) Pub. Date: Feb. 2, 2023

(54) PHALAENOPSIS PLANT NAMED '311NR'

(50) ;

(71) Applicant: ISHIHARA SANGYO KAISHA, LTD., Osaka (JP)

(72) Inventors: Shunji Yuki, Shiga (JP); Masahiro Mii, Tokyo (JP); Dong Poh Chin, Tokyo (JP)

(73) Assignee: ISHIHARA SANGYO KAISHA, LTD., Osaka (JP)

(21) Appl. No.: 17/683,638

(22) Filed: Mar. 1, 2022

(30) Foreign Application Priority Data
Aug. 2, 2021 (JP) 35624

(51) Int. Cl. A01H 6/62

(52) U.S. Cl. PLT/311

(57) ABSTRACT

‘311NR’ is a new and distinct variety of Phalaenopsis orchid; the instant variety is distinguishable from other cultivars by its dark purple flower petals, overall plant length, and width of flower and flower petals.

Latin name of the genus and species of the plant claimed: Phalaenopsis Blume.

Varietal denomination: ‘311NR’.

### RELATED APPLICATIONS

This application claims priority to Japanese Plant Variety Protection Application No.: 35624, filed Aug. 2, 2021, pursuant to 35 U.S.C. § 119(f).

### BACKGROUND OF THE NEW PLANT

The present invention comprises a new Phalaenopsis, botanically known as Phalaenopsis Blume and hereafter referred to by the new variety name ‘311NR’.

The candidate variety ‘311NR’ was produced, in part, by genetic recombination. The candidate variety was produced by introducing, to Phalaenopsis Wedding Promenade, F3′ 5′ H gene derived from Commelina communis.

To asexually reproduce ‘311NR’, axillary buds of flower stalks were cultured in a HYPONeX culture medium containing plant hormones (0.1 mg/l NAA, 1 mg/l BA) so that protocorm-like bodies (PLB) were induced and grown. Thereafter, PLB were redifferentiated in a hormone-free culture medium and flask seedlings were prepared.

‘311NR’ originates from a controlled breeding program at the Ishihara Sangyo Kaisha Ltd. Central Research Institute in Kusatsu, Shiga, Japan.

### BRIEF SUMMARY OF THE INVENTION

‘311NR’ has not been observed under all possible environmental conditions. The phenotype may vary significantly with variations in environment such as temperature, light intensity, and day length.

Plant Breeder's Rights for this cultivar have been applied for in Japan on Aug. 2, 2021. ‘311NR’ has not been made publicly available more than one year prior to the filing of this application.

The following traits have been repeatedly observed and are determined to be basic characteristics of the new variety. The combination of these characteristics distinguishes this Phalaenopsis as a new and distinct variety.

### BRIEF DESCRIPTION OF THE PHOTOGRAPHS

The accompanying photographic drawings show typical foliate characteristics of ‘311NR’ with colors being as true as possible with an illustration of this type.

FIG. 1 shows photographic drawings of cultivation of ‘311NR’ in the greenhouse location four years after planting.

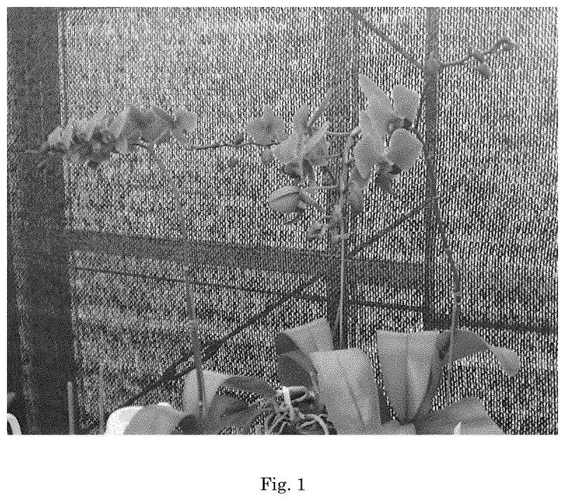

FIG. 2 shows an entire view of the plant two years after planting.

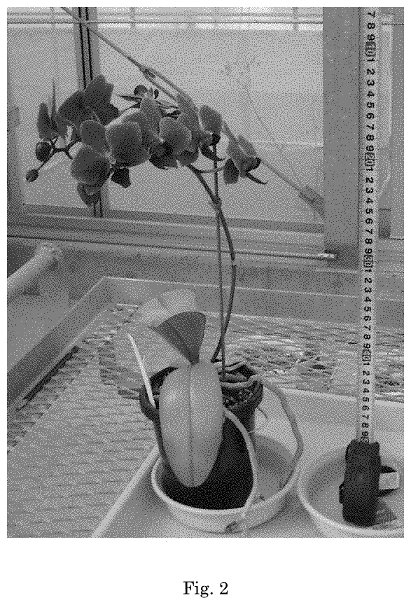

FIG. 3 shows inflorescence of the plant two years after planting.

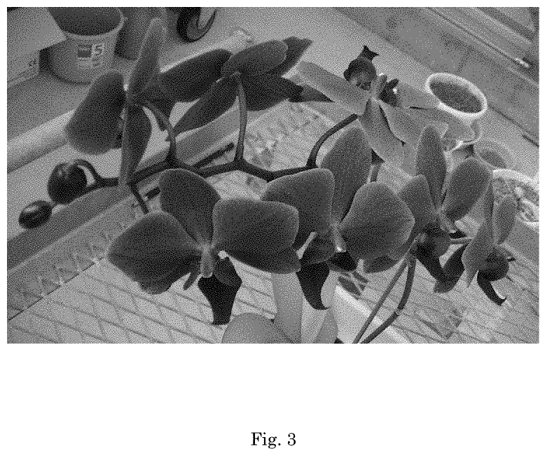

FIG. 4 shows an enlarged depiction of flowers from an aerial point of view two years after planting.

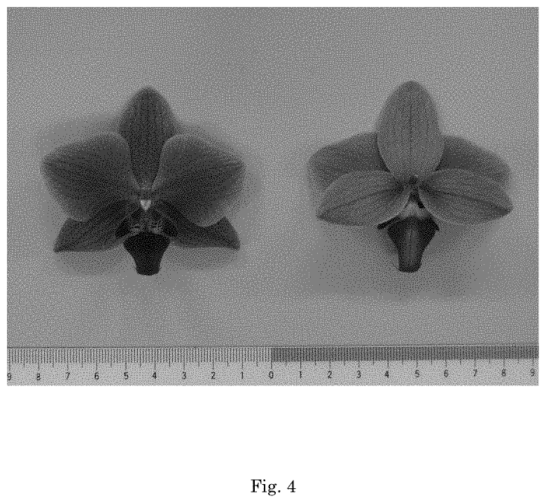

FIG. 5 shows an enlarged depiction of flowers from aerial and side points of view two years after planting.

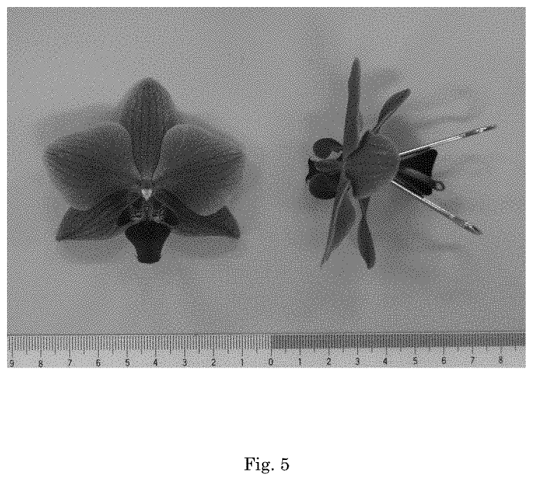

FIG. 6 shows an exploded depiction of a flower from an aerial view two years after planting.

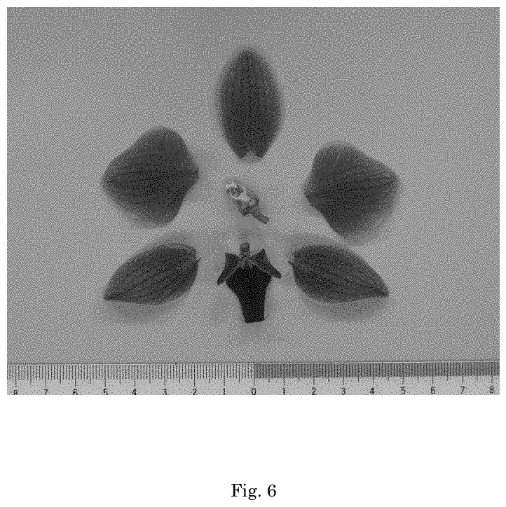

FIG. 7 shows a comparative depiction of entire plants ‘311NR’ and parent variety ‘Wedding Promenade’ two years after planting.

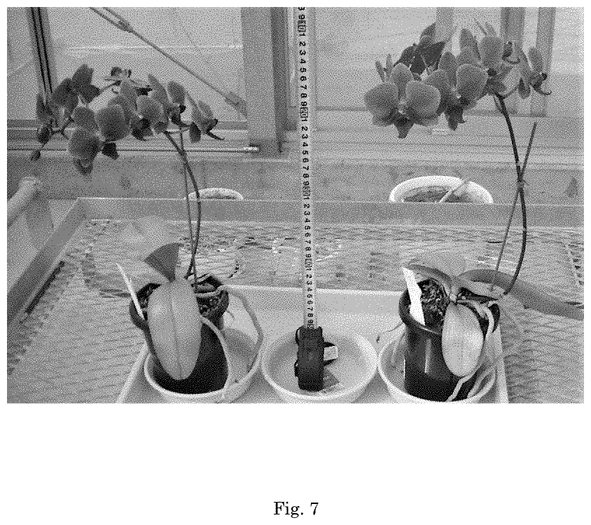

FIG. 8 shows a comparative depiction of flowers from ‘311NR’ and parent variety ‘Wedding Promenade’ from an aerial point of view two years after planting.

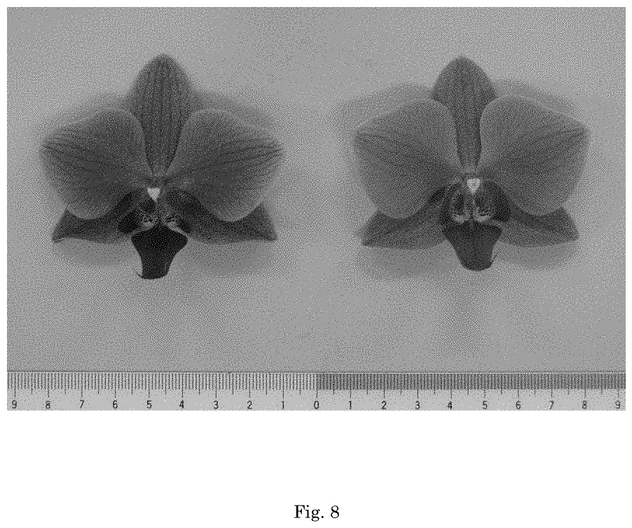

FIG. 9 shows a comparative depiction of entire plants ‘311NR’ and ‘Summer Rose’ approximately two years after planting.

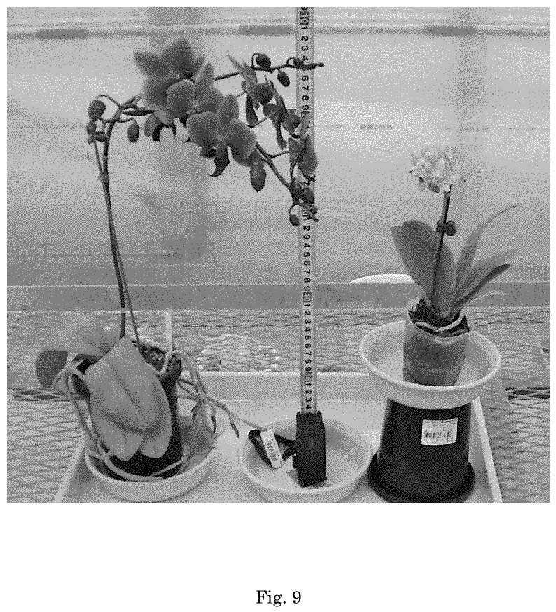

FIG. 10 shows a comparative depiction of flowers from ‘311NR’ and ‘Summer Rose’ from an aerial point of view approximately two years after planting.

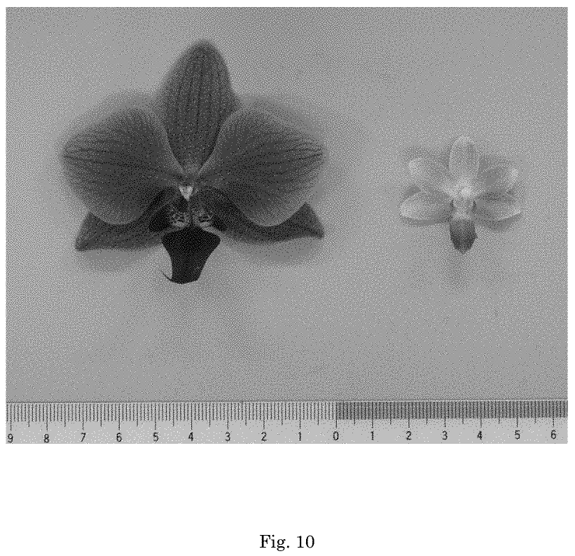

### DETAILED BOTANICAL DESCRIPTION

TABLE 1
DIFFERENCES BETWEEN THE NEW VARIETY AND
SIMILAR VARIETIES

| Name of Similar | Description of | Characteristics of | Characteristics |
|---|---|---|---|
| Variety | Characteristics | Similar Variety | of ‘311NR’ |
| Wedding | Petal: ground | N74D, N78D | 91A |
| Promenade | color of | | |
| | upper side | | |
| Wedding | Petal: over | N74B, N78B | N88A, N89A |
| Promenade | color | | |
| | (if present) | | |
| Wedding | Petal: color of | 75D | 91B |
| Promenade | spots | | |
| Wedding | Petal: color of | N74A, N78A | N89B |
| Promenade | stripes | | |
| Wedding | Petal: color of | N74A, N78A | N89B |
| Promenade | netting | | |
| Summer Rose | Plant: length | 26 cm | 51 cm |
| Summer Rose | Flower: width | 38 mm | 70 mm |
| | in front view | | |
| Summer Rose | Petal: ground | NN155C | 91A |
| | color of | | |
| | upper side | | |
| Summer Rose | Petal: over | N87B, N87C | N88A, N89A |
| | color | | |
| | (if present) | | |
| Summer Rose | Petal: color of | N87B, N87C | N89B |
| | stripes | | |

Color chart used: Royal Horticultural Society Colour Chart (R.H.S.) 5^{th} edition.

The following description is applied to our plants that are 24 months old as of the time of the measurements.
Plant:
   Length.—51 cm.
Leaf:
   Variegation.—Absent.
   Spots on upper side.—Absent.
Flower:
   Width in front view.—70 mm.
Petal:
   Ground color of upper side.—91A.
   Over color.—N88A, N89A.
   Number of spots.—Few.
   Color of spots.—91B.
   Number of stripes.—Medium.
   Color of stripes.—N89B.
   Density of netting.—Low.
   Color of netting.—N89B.
Other characteristics:
   Commercial classification.—Ornament, pot flower, or cut flower.
   Method of preservation and storage.—Cultured in pod in greenhouse.
   Time for seeding and planting.—Early January to later December.
   Growing stage, including flowering.—Middle February to late May.

What is claimed is:

1. A new and distinct variety of Phalaenopsis plant named ‘311NR’, substantially as illustrated and described herein.
